SEQUENCE LISTING (12 sequences)

SEQ ID NO: 1 - DNA, 21 bp, Homo sapiens
tcaggaggctgaatgtcagtt

SEQ ID NO: 2 - DNA, 23 bp, Homo sapiens
aaagccgctccgctcctcttcta

SEQ ID NO: 3 - DNA, 25 bp, Homo sapiens
cgtgcagcggtttagtttaatttcg

SEQ ID NO: 4 - DNA, 18 bp, Homo sapiens
aggcacctcccactcaca

SEQ ID NO: 5 - DNA, 20 bp, Homo sapiens
cacccgtgcacactcttggg

SEQ ID NO: 6 - DNA, 22 bp, Homo sapiens
cgactcccaagtcaccatcaag

SEQ ID NO: 7 - DNA, 19 bp, Homo sapiens
ggtgctgagccttgatgac

SEQ ID NO: 8 - DNA, 25 bp, Homo sapiens
tccctacagtggagatttccaagga

SEQ ID NO: 9 - DNA, 18 bp, Homo sapiens
tcccagagtcgcaggaaa

SEQ ID NO: 10 - DNA, 21 bp, Homo sapiens
ggagtgccctagaggatgaga

SEQ ID NO: 11 - DNA, 22 bp, Homo sapiens
tggctccctgtcaggttgaaca

SEQ ID NO: 12 - DNA, 23 bp, Homo sapiens
ggcagggactaataccactgatg

(19) United States
(12) Patent Application Publication
(10) Pub. No.: US 2013/0122499 A1
(43) Pub. Date: May 16, 2013

(54) SYSTEM AND METHOD OF DETECTING LOCAL COPY NUMBER VARIATION IN DNA SAMPLES

(71) Applicant: VIOMICS, INC., Phoenix, AZ (US)

(72) Inventors: Scott Morris, Phoenix, AZ (US); David Mallery, Solana Beach, CA (US)

(73) Assignee: VIOMICS, INC., Phoenix, AZ (US)

(21) Appl. No.: 13/675,743

(22) Filed: Nov. 13, 2012

Related U.S. Application Data
(60) Provisional application No. 61/559,589, filed on Nov. 14, 2011.

(51) Int. Cl. C12Q 1/68

(52) U.S. Cl. CPC C12Q 1/68
USPC 435/6.11

(57) ABSTRACT

Systems and methods for measuring local copy number variation in DNA samples are provided. In particular, methods for detecting copy number variation in circulating free DNA (cfDNA) that may be used to assay for copy number variations often corresponding to cancerous cells or tumors are provided.

### REFERENCE TO SEQUENCE LISTING

The present application is filed with a Sequence Listing in Electronic format. The Sequence Listing is provided as a file entitled VIOMC001A_SEQUENCE.txt, created Nov. 7, 2012, which is approximately 1.9 kb in size. The information in the electronic format of the sequence listing is incorporated herein by reference in its entirety.

### CROSS-REFERENCE TO RELATED APPLICATIONS

This application claims priority to U.S. Provisional Patent Application 61/559,589, filed on Nov. 14, 2011 and entitled SYSTEM AND METHOD OF DETECTING LOCAL COPY NUMBER VARIATION IN DNA SAMPLES, the entirety of which is hereby incorporated by reference herein.

### FIELD OF THE INVENTION

Systems and methods for measuring local copy number variation in DNA samples are provided. In particular, methods for detecting copy number variation in circulating free DNA (cfDNA) that may be used to assay for copy number variations often corresponding to cancerous cells or tumors are provided.

### BACKGROUND OF THE INVENTION

Human cells release circulating free DNA (cfDNA) into the bloodstream. This DNA is free DNA that is not contained within a cell. Although cfDNA may be present in low levels (i.e., 1-200 ng/μl) studies have shown that cfDNA may be enriched for tumor DNA. It is also known that tumors often develop copy number variants (CNVs) in certain genomic regions. For example, CNVs can correspond to regions of the genome that are either deleted or duplicated in certain chromosomes. Deletions in the genome result in reduced copy numbers of genes, while duplications in the genome result in increased copy numbers. In general, regions containing one or more cell-growth or cell-division-promoting genes, or other genes that promote tumor formation or growth, have increased copy number, while regions that contain tumor suppressor genes or that otherwise inhibit tumor formation or growth have decreased copy number. These CNVs tend to co-occur in similar locations within a tumor type, although there may be some variation in the CNVs observed between tumor types.

### SUMMARY OF THE INVENTION

The systems, devices, and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

In one embodiment, a method includes detecting copy number differences in DNA samples taken from a patient based on the copy number of regions that harbor copy number variants (CNVs). In one embodiment, a method includes detecting copy number differences in cfDNA. In one embodiment, real-time PCR is used as a detection method to determine the CNVs. Copy number variations may be indicative of cancerous cells, tumors, or cells with a heightened potential to become cancerous. In some embodiments, the methods include the use of Taqman® chemistry. In some embodiments, DNA sequencing may be used as a detection method to determine CNVs.

In one embodiment, the results of DNA copy number, mutation or methylation assays are analyzed to determine an assay outcome (i.e., positive or negative result) based at least in part on statistical distances between results. In some aspects, patients may be classified into different risk groups based at least in part on the analysis of CNVs as disclosed herein. The cumulative distribution function of the normal, binomial and/or Poisson distribution or similar functions may be used to determine CNVs. In some embodiments, the type of cancer present in a patient based may be predicted at least in part on results of DNA copy number, mutation or methylation analysis alone or in conjunction with clinical, demographic or lifestyle attributes that utilize, for example, Bayes' theorem.

In some embodiments, a method of detecting copy number variation indicative of cancer or a likelihood of cancer in a nucleic acid sample obtained from a subject is disclosed. An aspect of these embodiments comprises identifying a first locus corresponding to a first gene, wherein the copy number of the first locus increases in some cancer types. An aspect of these embodiments comprises identifying a second locus corresponding to a second gene, wherein the copy number of the second locus decreases in some cancer types. An aspect of these embodiments comprises obtaining a deoxyribonucleic acid sample from said subject. An aspect of these embodiments comprises assaying the copy number of said first locus. An aspect of these embodiments comprises assaying the copy number of said locus. An aspect of these embodiments comprises calculating a ratio of the copy number of said first locus to the copy number of said second locus in said nucleic acid sample, wherein said ratio is indicative of a cancer status in said subject.

An aspect of these embodiments comprises performing at least one quantitative polymerase chain reaction.

An aspect of these embodiments comprises a sample comprising circulating free deoxyribonucleic acid.

In an aspect of these embodiments, the cancer assayed is lung cancer, such as lung adenocarcinoma.

In an aspect of these embodiments, a first locus corresponds to a gene selected from the list consisting of: cMYC, NKX2-1 and EFGR. In an aspect of these embodiments, a second locus corresponds to CDK2Na.

In some embodiments, a method of assaying for a genetic signature indicative of cancer in a patient is disclosed. An aspect of these embodiments comprises obtaining a blood sample from said patient.

An aspect of these embodiments comprises isolating circulating free DNA from said sample.

An aspect of these embodiments comprises determining the copy number of at least a first genetic region, wherein an increased copy number is indicative of cancer in a patient. An aspect of these embodiments comprises determining the copy number of at least a second genetic region, wherein a decreased copy number is indicative of cancer. An aspect of these embodiments comprises determining the ratio of the copy number of said first genetic region to the copy number of said second genetic region. An aspect of these embodiments comprises identifying said sample as having said genetic signature indicative of cancer if said ratio is above a threshold value. In some aspects of these embodiments a copy number of a first region is below a threshold proportional increase value indicative of cancer; a copy number of a second region is below a threshold proportional decrease value indicative of cancer; and a ratio of the copy number of a first genetic region to the copy number of a second genetic region is above a threshold value indicative of cancer, such that a signal indicative of cancer or precancerous DNA is detectable.

### DETAILED DESCRIPTION

When applied to large-scale genomics projects such as The Cancer Genome Atlas (TCGA), common CNVs may be readily identified for multiple cancer types. Once CNVs are known within a cancer type, similar CNVs may exist in the cfDNA of plasma taken from cancer patients. Because plasma can be collected and prepared within many primary care physician offices without posing any more risk than a standard blood draw, cfDNA CNVs may be a valuable cancer biomarker. Additionally, if cfDNA CVNs may be assayed reliably, they may have a number of advantages over current cancer assays. For example, CNVs may detect cancer at an early stage of development, or cancer that may be developing in an area of the body that may not be accessible to traditional biopsy assays.

Current methods of determining CNVs rely on a fixed cutoff to measure CNV relative to healthy or copy-number invariant DNA as a control. Such fixed-cutoff methods may rely on Receiver Operating Characteristic (ROC) curves to determine the presence of significant CNV in a sample. Current assays to detect cfDNA CNV can include qPCR and dPCR. However, these techniques may not be sufficiently specific to detect the CNV resulting from very small populations of cancer cells which may represent tumors in very early stages of development.

Thus, one embodiment is an assay that determines the ratio of the copy number of a high copy number variant to the copy number of a low copy number variant in a patient, for example, in a sample derived from a patient such as a blood sample comprising circulating free DNA. As used herein, a high copy number variant locus is a locus which increases in copy number in at least some forms of cancerous cells as compared to noncancerous cells. As used herein, a low copy number variant locus is a locus which decreases in copy number in at least some forms of cancerous cells as compared to noncancerous cells.

In some embodiments, low copy number means less than 2 copies within at least some cells of at least part of a tumor (i.e., 1 or zero copies in some tumor cells). In some embodiments, high copy number means more than 2 copies within at least some cells of at least part of a tumor. For example, the copy number can be 3 in some tumor cells, but in certain genes such as MYC the copy number can increase in some tumor cells in at least parts of a tumor to more than 10 copies.

In some embodiments copy number variant loci comprise nucleic acids having sequences which are partially or completely repetitive. Embodiments of the invention relate to using the ratio of the copy number of a high copy number variant to the copy number of a low copy number variant. This analysis may be based on a statistical distance calculation rather than a fixed cutoff determination using a ROC curve. Cancer cells and precancerous cells may be characterized by increased copy numbers of genes in the vicinity of cancer promoting genes relative to the copy numbers of homologous loci in noncancerous cells of the same individual. In addition, cancer cells and precancerous cells may be characterized by a decreased copy number of DNA at other loci. In some cases the CNV may be in cancer-inhibiting genes or loci in the vicinity of cancer inhibiting genes relative to the copy numbers of homologous loci in noncancerous cells of the same individual.

Many cancers show both an increase in some copy numbers and a decrease in other copy numbers. Thus, in one embodiment, the copy number of CNV genes having cancer promoting and cancer-blocking properties may be used to assay for CNVs indicative of cancer by measuring the ratio of two copy number variants, one a high copy number variant and the second a low copy number variant to one another. In one embodiment, the ratio is represented as a statistical distance between the opposing CNV values.

By assaying for the ratio of separate loci, which may change inversely to one another in cancerous cells (with copy number at one increasing while the copy number at the second decreases, thus increasing the statistical distance), embodiments of this invention detect with statistical confidence cfDNA CNV that may be below the threshold of detection of other methods. Through this method, some embodiments may be able to detect cfDNA signals which may be too weak to be detected by state of the art assays currently available.

In some embodiments, the types of cancers assayed may include cancers of the following organs and types: lung, pancreas, esophagus, colon, stomach, breast, prostate, thyroid, bladder, kidney, head, neck, uterine, leukemia, non-Hodgkin lymphoma, liver, ovary, cervix. In one embodiment, the cancer assayed may include non-small cell lung cancer.

Although this disclosure is primarily directed to cancer detection, embodiments need not be limited to this topic. Any DNA CNV may be assayed by embodiments disclosed herein, independent of the cellular process to which they may be related, the cause behind the CNV assayed or the source of DNA used in the CNV assay.

Drug targets may be assayed in some embodiments. Some embodiments may, for example, amplify HER2 cfDNA for evaluation of effectiveness of treatment by, for example, Trastuzumab, or amplify EGFR cfDNA for evaluation of effectiveness of treatment by, for example, Gefitinib, Erlotinib, Cetuximab, or Panitumumab, or amplify AKT1 cfDNA for evaluation of effectiveness of treatment by, for example cisplatin, or amplify VEGF cfDNA for evaluation of effectiveness of treatment by, for example, Vandetanib.

In one embodiment the method can begin by extracting cfDNA from a biological sample taken from a patient. In some embodiments, a consistent, repeatable method is used to isolate cfDNA from plasma or other source of DNA to ensure the reliability of the data. To obtain cfDNA from patient blood, one may use the protocol listed below although other methods are also contemplated

cfDNA molecules may be purified from plasma or other samples using, for example, Qiagen's QIAamp circulating nucleic acid kit. The protocol in this kit provides an embodiment of a method to purify circulating total nucleic acid from 1 mL of plasma. Samples produced by this method may be highly pure and free of PCR inhibitors, and may be suitable for qPCR as used in some embodiments to assay cfDNA to determine the copy number of one or more high copy number loci and one or more low copy number loci. Analyzing the ratio of the copy numbers of at least one high copy number locus to at least one low copy number locus, one can calculate statistical distances as an assay of, for example, nucleic acids present in the circulating free DNA of a patient indicative of various types of cancer.

In one embodiment the method may involve PCR amplification of cfDNA template DNA. cfDNA may be amplified by, for example, a consistent, repeatable method to amplify cfDNA from plasma or other DNA. This protocol may not be the only suitable protocol to amplify cfDNA. However, it may be important to use a consistent protocol for cfDNA amplification, as variations in protocol may have a large effect on the eventual results.

Plasma contains circulating free DNA and RNA (cfDNA and cfRNA). These circulating nucleic acids originate from multiple tissues, but tend to be enriched for cancer-derived DNA when cancer is present in a patient. An embodiment of the invention involves a standardized method to detect extremely dilute DNA with high levels of accuracy. When the results of this assay are combined with Viomics' proprietary algorithms, tumor markers can be detected with high levels of confidence.

In one embodiment the method may involve an assay for lung adenocarcinoma, such as the U2a assay for lung adenocarcinoma and other cancer CNVs. A proprietary U2a assay is provided. In this embodiment, the assay may be a 4-plex qPCR assay that detects focal regions of copy number variation near CDK2Na, cMYC, NKX2-1 and EGFR, four loci which undergo CNV in lung adenocarcinomas. Note that the genes listed here are only for ease of reading. The actual markers fall within genomic regions that are near to these genes. CDK2Na may be the most common gene to experience loss of copy number in lung adenocarcinoma, and the other three genes may be the most likely to gain copy number.

The U2a assay components are listed below in Table 1.

TABLE 1

| Nearby | | | | |
|---|---|---|---|---|
| Gene | Type | Forward Primer | Probe | Reverse Primer |
| uCDKN2A-2 | Loss | TCAGGAGGCTGAATGTCAGTT | FAM-AAAGCCGCTCCGCTCCTCTTCTA-BHQ1 | CGTGCAGCGGTTTAGTTTAATTTCG |
| | | (SEQ ID NO: 1) | (SEQ ID NO: 2) | (SEQ ID NO: 3) |
| uMYC | Gain | AGGCACCTCCCACTCACA | Cal fluor gold 520- | CGACTCCCAAGTCACCATCAAG |
| | | (SEQ ID NO: 4) | CACCCGTGCACACTCTTGGG-BHQ1 | (SEQ ID NO: 6) |
| | | | (SEQ ID NO: 5) | |
| uNKX2-1 | Gain | GGTGCTGAGCCTTGATGAC | Cal fluor red 610- | TCCCAGAGTCGCAGGAAA |
| | | (SEQ ID NO: 7) | TCCCTACAGTGGAGATTTCCAAGGA-BHQ2 | (SEQ ID NO: 9) |
| | | | (SEQ ID NO: 8) | |
| uEGFR | Gain | GGAGTGCCCTAGAGGATGAGA | Quasar | GGCAGGGACTAATACCACTGATG |
| | | (SEQ ID NO: 10) | 705-TGGCTCCCTGTCAGGTTGAACA-BHQ3 | (SEQ ID NO: 12) |
| | | | (SEQ ID NO: 11) | |

All sequences are listed in the 5′ to 3′ orientation, although actual 5′ and 3′ ends may be masked by, for example, detection molecule components such as, for example, Cal fluor gold 520 or BHQ1. Other detection molecule components may be used, including, for example, FAM, Cal fluor gold 540, Cal fluor red 610, quasar 705, or others. The final concentrations of reagents in the U2a assay may be 0.1 μM of each primer and 0.04 μM of each probe. Of these assay components and targets, uMYC may be the best gain marker. uNKX2-1 was not used in result calculation. Standards used with these components may include a high standard of 3 ng/μL, and a low standard of 0.3 ng/μL.

Assays may involve components of different sequence or with different detectable labels targeted to similar regions, components targeted to different regions of the same genes, or components targeting the regions of genes other than those listed in the U2a assay above.

The results of a U2a test may be evaluated using the Decision Rules for Viomics' Lung Adenocarcinoma Test, given below. The results are calculated for 95% specificity, and may use the following criteria: if <0.5 ng total DNA or >200 ng total DNA, then no result; if 0.5-5 ng total DNA then the result may be scored as negative; if 30 ng-200ng then the result may be scored as positive; if 5 ng-30 ng, Table 2 below may be used.

TABLE 2

| Ref | | Copies | | | |
|---|---|---|---|---|---|
| DNA | | (4 × 7.5 uL | | | Min Ratio |
| Conc. | ng/mL | rxn) | Possion CV | Assay CV | for positive |
| 0.070 | 4.9 | 305.2 | 0.0572 | 0.04 | 1.1770 |
| 0.080 | 5.6 | 348.8 | 0.0535 | 0.04 | 1.1678 |
| 0.090 | 6.3 | 392.4 | 0.0505 | 0.04 | 1.1604 |
| 0.100 | 7 | 436.0 | 0.0479 | 0.04 | 1.1542 |
| 0.120 | 8.4 | 523.3 | 0.0437 | 0.04 | 1.1443 |
| 0.140 | 9.8 | 610.5 | 0.0405 | 0.04 | 1.1369 |
| 0.160 | 11.2 | 697.7 | 0.0379 | 0.04 | 1.1310 |
| 0.180 | 12.6 | 784.9 | 0.0357 | 0.04 | 1.1262 |
| 0.200 | 14 | 872.1 | 0.0339 | 0.04 | 1.1223 |
| 0.220 | 15.4 | 959.3 | 0.0323 | 0.04 | 1.1190 |
| 0.240 | 16.8 | 1046.5 | 0.0309 | 0.04 | 1.1161 |
| 0.260 | 18.2 | 1133.7 | 0.0297 | 0.04 | 1.1137 |
| 0.280 | 19.6 | 1220.9 | 0.0286 | 0.04 | 1.1115 |

In one embodiment the method may involve a Statistical Distance Determination. Because cfDNA from cancer cells may be highly diluted by normal cfDNA, a method may be required to determine significant changes in copy number. For this reason, in one embodiment, the method determines the assay outcome (i.e., positive or negative result) based on statistical distances between results as opposed to a fixed cutoff determined only through ROC curves. One embodiment of a method by which this statistical distance may be calculated is discussed below.

Through the use of the methods taught herein, one may be able to detect cancer signatures in cfDNA wherein a decrease in low copy number gene level has occurred, and an increase in a high copy number gene level has occurred in a cfDNA sample, and wherein the statistical signal generated by comparing the level of one to the other is sufficient to identify a cancer signature in said sample even if the statistical signal generated by either event (the increase or the decrease alone) is insufficient to warrant a cancer determination on its own.

In one embodiment the method may involve Sample Models and Derivations to analyze DNA copy number results. In one embodiment, a cancer-free individual does not have any CNVs between different DNA fragments in cfDNA. Thus, using this embodiment, the hypothesis is that the individual is cancer-free and the expected number of copies of each gene may be equal. In order to accept or reject this hypothesis, an understanding of the underlying distributions that describe the number of DNA fragments that may be detected by the assay is derived.

This method begins by separating variance into components: detection variance (σ²_{D}) and biological variance (σ²_{B}). In this embodiment both variance types can be estimated by the normal distribution via the normal distribution via the central limit theorem, so the total variance, σ², can be expressed as:

σ²=σ²_{D}+σ²_{B} (1)

In this embodiment, biological variance is first evaluated. If each cell can release cfDNA once within a predetermined amount of time, the number of copies of a given fragment follows the binomial distribution, with n being the number of cells and p being the probability of a given cell releasing DNA into the plasma. In this embodiment, n may be on the order of 10¹³ cells in the human body. In this embodiment there may be about 6 ng of DNA per mL plasma, or 1000 genomes per mL plasma. Thus an estimate of p for a single mL of plasma in this embodiment may be on the order of 10⁻¹⁰.

Considering the large value of n and the small value of p, one may use the Poisson Distribution in this embodiment to model biological variance:

P(K=k)=(μ^{k}e^{-k})/k! (2)

The Poisson distribution may give an advantage because it has a single parameter, μ, which represents the number of fragments present. This single parameter can be used to describe both expected value and variance. Thus one may estimate both these statistics from a single measurement, or one may estimate variance based on the arithmetic mean with much higher accuracy that the S statistic. The coefficient of variation (CV) for the biological variance can be calculated as:

c_{B}=1/√(\hat{μ}) (3)

The detection variance can then be evaluated. This detection variance follows a normal distribution, multiplied by some correction factor, a, i.e. N (a, a²σ²_{D}). Provided that parameters a and σ² may be determined experimentally and known in advance, CV for detection may be calculated as:

c_{D}=[√(α²σ²)]/α=σ_{D} (4)

A meaningful distance measurement can then be determined between reads. In this embodiment the central limit theorem allows one to estimate biological variance with the standard normal distribution. Because μ>>σ, in this embodiment the central limit theorem applies to the CV as well. In a cancer-free individual in this embodiment, the copy number of all genes may be equal. Thus, the CV of each marker can be estimated as a function of \hat{μ}, the estimated number of fragments present:

c(\hat{μ})=√(c²_{B}+c²_{D})=√(1/\hat{μ}+σ²_{D}) (5)

When one considers the option of multiple runs on the machine for each specimen, and given that in this embodiment the sample contains some finite number of DNA fragments that are divided amongst each run, one may calculate the CV of the average value of n runs in this embodiment as:

c(\hat{μ}, n)=√(c²_{D}+c²_{B})=√(1/\hat{μ}+σ²_{D}/n) (6)

The value of cₙₑₜ will be calculated individually for each specimen. Note that the device variance may be reduced by increasing n, but biological variance does not change.

Now, some sample calculations can be conducted using estimates of 20 ng of DNA per sample from a healthy individual ≈3000 fragments, 4 replicates and C_{D}=0:06 (determined by, for example, conducting the U2a assay on blood).

c=√( 1/3000+(0.06)²/4)=3.51% (7)

Next, in this embodiment one has a reference gene (i.e. one that loses copy number in cancer) and a target gene (i.e. one that gains copy number in cancer). One may subtract the reference gene mean from the target gene mean in this embodiment to find the difference (i.e. μₜₐᵣ−μ_{ref}). One may now calculate the CV of this comparison, which in the given example with certain known values of variance and c_{ref}=cₜₐᵣ is as follows:

cₙₑₜ=√(c²_{ref}+c²ₜₐᵣ)=c√2=4.95% (8)

In the worst case that would create a detectable event, either the target gene or the reference gene would experience a CNV. If both the reference gene and a target gene experienced a CNV, it would be easier to detect. One may define τ to be the threshold for calling a result significant. τ can be calculated as a function of α, our target sensitivity, using the Z distribution for a one-tailed test:

α(τ)=Z(τ/cₙₑₜ) (9)

Tau will be calculated once and will be used for all samples. In one embodiment, one may call a differences of τ=1.82 standard deviations (cₙₑₜ) as significant. Using Bonferroni correction for 3 comparisons, one may estimate that this will give us a specificity of 90%. Thus, the minimal detectable event at 50% sensitivity is:

τcₙₑₜ=1.82cₙₑₜ=9% (10)

In general, the power function for some read from a “gain” marker, μ′, can be derived from the Z distribution:

β(μ)=Z[((μ′−μ)/(μ−τ))/cₙₑₜ] (11)

For example, one may be able to detect a 15% difference in copy number 89% of the time, and a 20% difference 99% of the time.

In one embodiment the method may involve Models and Derivations for classification of patients into risk groups. In a further embodiment of the invention, medical professionals or other users of the invention may want to classify patients into risk groups based on the CNV results ascertained using the embodiments above. Such classification may be accomplished using the techniques below.

Based on the previous analysis, it may be quite simple to calculate the specificity as the α value from above.

β(μ)=Z[((μ′−μ)/μ)/cₙₑₜ] (12)

Based on the specificity, the results can be divided into groups (high confidence, low confidence, etc.). This number may also be transformed by some simple formula to create a numerical score for confidence.

In one embodiment the method may involve Models and Derivations for predicting the type of cancer present in a patient based on results of DNA copy number, mutation or methylation analysis in conjunction with some clinical, demographic or lifestyle attribute(s)

In a further embodiment of the invention, medical professionals or other users of the invention may want to predict the type of cancer present in a patient based on the CNV analysis results in conjunction with some clinical, demographic or lifestyle attribute(s). Such classification may be accomplished using the techniques below.

Bayes' theorem allows calculation of probabilities conditional on other known events. For example, knowledge that a patient is a smoker increases both the probability that the patient has cancer, and increases the conditional probability that the cancer originates in the lung. Likewise, being female will substantially increase the risk that the cancer originates in the breast vs. somewhere else. The general form of Bayes' theorem is:

P(A|B)=P(AB)/P(B)=P(B|A)P(A)/P(B) (13)

Using multiple levels of conditioning, one may condition on multiple events to predict the probability that a patient has a certain type of cancer. Examples of events that can be used in conditioning include the following: results from, for example, a U2a test; clinical attributes; results from other tests, X-ray, symptoms, other conditions; lifestyle attributes, such as smoking history, sun exposure, or other attributes that may increase the risk of cancer; or demographics, such as gender, age, or ethnicity. Other events or characteristics may be used as well or alternatively. Additional uses of Bayes' theorem may include Bayesian networks and Naive Bayes classifiers.

What is claimed is:

1. A method of detecting copy number variation indicative of cancer in a nucleic acid sample obtained from a subject comprising:
identifying a first locus corresponding to a first gene, wherein the copy number of the first locus increases in some cancer types,
identifying a second locus corresponding to a second gene, wherein the copy number of the second locus decreases in some cancer types,
obtaining a deoxyribonucleic acid sample from said subject,
assaying the copy number of said first locus,
assaying the copy number of said locus,
calculating a ratio of the copy number of said first locus to the copy number of said second locus in said nucleic acid sample, wherein said ratio is indicative of a cancer status in said subject.

2. The method of claim 1, wherein said assaying comprises performing at least one quantitative polymerase chain reaction.

3. The method of claim 1, wherein said sample comprises circulating free deoxyribonucleic acid.

4. The method of claim 1, wherein said cancer is lung cancer.

5. The method of claim 1, wherein said cancer is lung adenocarcinoma.

6. The method of claim 1, wherein said first locus corresponds to a gene selected from the list consisting of: cMYC, NKX2-1 and EFGR.

7. The method of claim 1, wherein said second locus corresponds to CDK2Na.

8. A method of assaying for a genetic signature indicative of cancer in a patient comprising:
obtaining a blood sample from said patient;
isolating circulating free DNA from said sample;
determining the copy number of at least a first genetic region, wherein an increased copy number is indicative of cancer in a patient;
determining the copy number of at least a second genetic region, wherein a decreased copy number is indicative of cancer;
determining the ratio of the copy number of said first genetic region to the copy number of said second genetic region; and
identifying said sample as having said genetic signature indicative of cancer if said ratio is above a threshold value.

9. The method of claim 8, wherein said copy number of said first region is below a threshold proportional increase value indicative of cancer; wherein said copy number of said second region is below a threshold proportional decrease value indicative of cancer; and wherein said ratio of the copy number of said first genetic region to the copy number of said second genetic region is above a threshold value indicative of cancer, such that a signal indicative of cancer or precancerous DNA is detectable.
